(19) United States
(12) Patent Application Publication
(10) Pub. No.: US 2004/0172008 A1
(43) Pub. Date: Sep. 2, 2004

(54) Hemostasis valve and method of using a hemostasis valve

(75) Inventor: James H. Layer, Cooper City, FL (US)

Correspondence Address: GMP COMPANIES, INC., ONE EAST BROWARD BLVD., SUITE 1701, FORT LAUDERDALE, FL, 33301, US

(73) Assignee: GMP/Cardiac Care, Inc., Fort Lauderdale, FL

(21) Appl. No.: 10/717,368

(22) Filed: Nov. 19, 2003

Related U.S. Application Data
(60) Provisional application No. 60/427,251, filed on Nov. 19, 2002.

(51) Int. Cl. A61M 25/16

(52) U.S. Cl. 604/533

(57) ABSTRACT

A hemostasis valve and a method of using the hemostasis valve to form a seal around a medical instrument that has been inserted through at least a portion of the valve. The hemostasis valve comprises a valve body having a proximal end for connecting to a first medical device and a distal end for connecting to a second medical device. The hemostasis valve also includes a first elongated chamber positioned within the valve body. A collapsible member positioned within the valve body defines this first elongated chamber. The first chamber has a first internal volume and is capable of receiving a medical instrument. The hemostasis valve additionally comprises a second elongated chamber extending about the first elongated chamber within the valve body. The second elongated chamber has an internal volume that is greater than the first internal volume. The hemostasis valve also includes a pressure application system comprising a member moveable within the second elongate chamber for increasing the pressure within the second elongate chamber and sealing the collapsible member about the received medical instrument.

### CROSS-REFERENCES TO RELATED APPLICATIONS

This application claims the benefit of U.S. Provisional Patent Application No. 60/427,251, filed on Nov. 19, 2002, the full disclosure of which is incorporated herein by reference.

### FIELD OF THE INVENTION

This invention relates generally to an apparatus that can be used to limit or prevent the loss of bodily fluids from a patient when an access device is introduced into the body of a patient, and more particularly to hemostasis valves used in diagnostic, therapeutic and interventional medical procedures.

### BACKGROUND OF THE INVENTION

There are many types of medical devices that are inserted into a patient's body, such as tubes, catheters, needles, trocars or other introducer sheathes and the like, through which catheters, needles or other medical devices can be introduced into a patient's body in order to perform a medical operation. As used herein, the term “catheter” is intended to embrace within its scope all of the above-mentioned medical devices and any device through which fluids are intended to be injected into the body of a patient or are removed from the body of a patient either intentionally or by accident, including by way of example but not limitation, tubes, catheters, needles, trocars or other introducer sheathes.

Hemostasis valves are well known and used in medical procedures requiring the insertion of a catheter into the vascular system of a patient. Hemostasis valves are employed for leak-proof introduction of catheters into the circulatory system of a patient or elsewhere in the body of the patient. Typically, a guide catheter is connected to the distal end of the hemostasis valve, and an operating instrument, such as a guide wire or balloon dilation catheter, is inserted into the proximal end and through the guide catheter to the desired location in the patient. After the operating instrument is in place, the valve is closed to prevent blood from escaping from the body of the patient. Hemostasis valves prevent the leakage of blood out of the ends of dilatation and guide catheters, to prevent the flow of blood between an inserted guide wire and the dilatation catheter, and also between the dilation catheter and the guide catheter.

One of the problems with some conventional hemostasis valves is that they are cumbersome to operate, taking a long time to open and close. Many of these conventional valves employ a Touhy-Borst sealing mechanism such as that described in U.S. Pat. No. 4,886,507. These conventional threaded caps deform an O-ring into a tapered opening until the O-ring clamps down on the operating instrument. Each time the operating instrument is adjusted, the cap must first be unthreaded to allow for the adjustment, and then subsequently rethreaded to reestablish the seal after the adjustment. During the time that the valve is open, blood and other fluids leak from the patient. Inaccurate blood pressure readings also occur. Further, these conventional valves present the risk of air emboli when the valve is open, particularly when removing the operating instrument.

Another problem with prior art hemostasis valves, such as Touhy-Borst valves, is that significant mechanical force must be applied to the operating instrument in order to maintain the seal. This is particularly a problem at higher system pressures, and when pressure spikes occur, such as when flushing the system with saline or introducing contrast media. The often delicate drive shaft of the operating instrument can be crushed by the force of the seal. The high force of the seal also prevents moving the operating instrument while the valve is closed. Additionally, the procedure required to apply the mechanical force can distract the surgeon and/or an attendant by requiring the use of at least two hands to accomplish the operation of the seal. The need for multiple hands to enter the surgical site to perform a single task can unnecessarily crowd the surgical site and possibly affect the performance and response of the surgeon. As a result, the operation can be jeopardized by a complicated valve structure that takes numerous hands to operate.

### SUMMARY OF THE INVENTION

Aspects of the present invention include a hemostasis valve and a method of using a hemostasis valve that overcome the disadvantages of the prior art hemostasis valves. These aspects of the invention can be used in a variety of diagnostic, therapeutic and interventional procedures, including, but not limited to angiography, angioplasty, stent placement, drug infusion, intravascular ultrasound, rotablation and atherectomy.

In one aspect of the invention, the hemostasis valve comprises a valve body having a proximal end for connecting to a first medical device and a distal end for connecting to a second medical device. The hemostasis valve includes a first elongated chamber positioned within the valve body. A collapsible member positioned within the valve body defines this first elongated chamber. The first chamber has a first internal volume and is capable of receiving a medical instrument. The hemostasis valve additionally comprises a second elongated chamber extending about the first elongated chamber within the valve body. The second elongated chamber has an internal volume that is greater than the first internal volume. The hemostatic valve also includes a pressure application system comprising a member moveable within the second elongate chamber for increasing the pressure within the second elongate chamber and sealing the collapsible member about a received medical instrument.

In one embodiment, the valve body includes a second chamber with a substantially hourglass shaped profile that creates a seal with the inner surface of the housing of the valve body. This self-forming seal prevents the need for sealing rings to be used with the element that reduces the volume within the larger chamber.

Another aspect of the invention includes a method of sealing a hemostasis valve about a medical instrument. The method comprises the steps of positioning a medical instrument within a first chamber in a valve body of the hemostasis valve, and advancing a pressure increasing element within a second chamber of the valve body. The second chamber surrounds at least a portion of the first chamber.

The sealing systems of the present invention eliminate the externally applied mechanical force devices that are commonly used to seal conventional hemostasis valves. As a result, the risk of damaging the operating instrument is significantly reduced and manipulation of operating instrument, longitudinally and torsionally, is permitted without destroying the seal about instrument.

The hemostasis valve according to the present invention can be carried by any catheter or sheath introducer, to permit an inner catheter, probe, or the like to be placed through the hemostasis valve to form a leak-proof seal and a port of entry. These and additional advantages and features of the invention are clear when the attached figures are viewed in light of the accompanying descriptive matter.

### BRIEF DESCRIPTION OF THE DRAWINGS

FIG. 1 is an elevational view of a first embodiment of a hemostasis valve according to the present invention;

FIG. 2 is a cross section of the hemostasis valve of FIG. 1;

FIG. 3 is a schematic drawing of the hemostasis valve of FIG. 1 without the plunger disk;

FIG. 4 is a perspective cross section of the hemostasis valve of FIG. 1;

FIG. 5 is an elevational view of a second embodiment of a hemostasis valve according to the present invention;

FIG. 6 is a cross section of the hemostasis valve of FIG. 5 with the plunger at rest;

FIG. 7 is a cross section of the hemostasis valve of FIG. 5 with the plunger in its pressure application position;

FIG. 8 is a schematic drawing of the hemostasis valve of FIG. 5;

FIG. 9 is a perspective view of a cross section of the hemostasis valve of FIG. 5;

FIG. 10 is a schematic drawing of a collapsible sealing member according to the present invention;

FIG. 11 is an elevational view of the collapsible sealing member;

FIG. 12 is cutaway, partial perspective view of the collapsible sealing member positioned within a valve body;

FIG. 13 is a schematic drawing of the hemostasis valve of FIG. 3 with a system for changing pressure within a chamber in response to a pressure increase in a catheter system; and

FIG. 14 is an isolated view of FIG. 13.

### DETAILED DESCRIPTION OF THE PREFERRED EMBODIMENTS

Referring now to the drawings, the same numerals are used to identify like parts of the illustrated embodiments. The hemostasis valves discussed herein can be used with any of the known diagnostic, therapeutic, and interventional medical instruments discussed above or similar instruments.

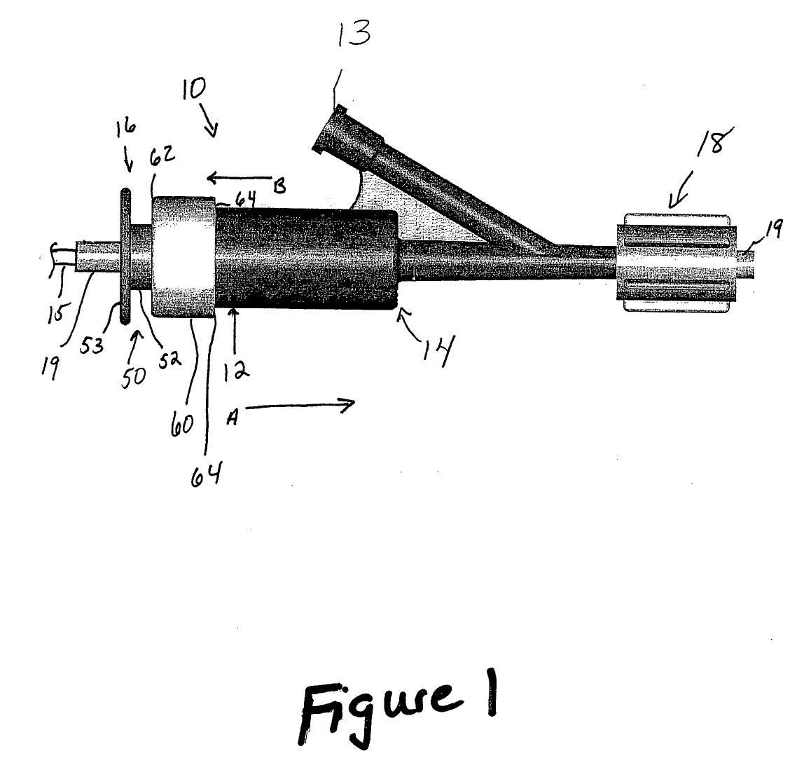

FIGS. 1-4 illustrate a hemostasis valve 10 according to the present invention. The hemostasis valve 10 comprises a valve body 12 that receives an internally inserted medical operating instrument 15, such as a guide wire or dilation catheter, such as a balloon catheter, that can move within the valve body 12 in a direction parallel to its longitudinal axis. The valve body 12 also includes an injection port 13 and conventional connectors at its proximal and distal ends 14, 16 for securing the valve body 12 to other instruments and devices used during a medical procedure. In a preferred embodiment, the proximal end 14 includes a conventional connector 18, such as a set of threads or a hose barb. In a preferred embodiment, the distal end 16 of the valve body 12, located opposite the proximal end 14, includes a standard luer lock (not shown) for connecting the valve body 12 to a guide catheter or other known catheters and medical instruments used in diagnostic, therapeutic and/or interventional medical procedures. The valve body 12 according to the present invention is not limited to these illustrated connectors. Instead, any known connector for securing two instruments together could be used at the proximal and distal ends 14, 16 of the valve body 12.

The valve body 12 includes an outer fluid carrying chamber 40 with an internal volume. The valve body 12 also includes a centrally positioned and longitudinally extending member 19 having a through-lumen 20 in which the operating instrument 15 is received and within which the operating instrument 15 moves. As illustrated in the figures, chamber 40 is not in fluid communication with through-lumen 20, but is instead isolated. Saline or another known fluid is provided to chamber 40 by a high pressure fluid source through a port (not shown). Additional ports may be included for pressure monitoring, flushing, and/or injecting contrast media for example.

As illustrated, the through-lumen 20 extends through the chamber 40 and beyond the terminal ends 14, 16 of the valve body 12. The through-lumen 20 includes an open section 21 that extends between two intermediate terminal portions 22, 23 of the elongated member 19 and defines and inner chamber 25 that surrounds an exposed portion of the operating instrument 15. The inner chamber 25 has an internal volume that is less than that of the outer chamber 40.

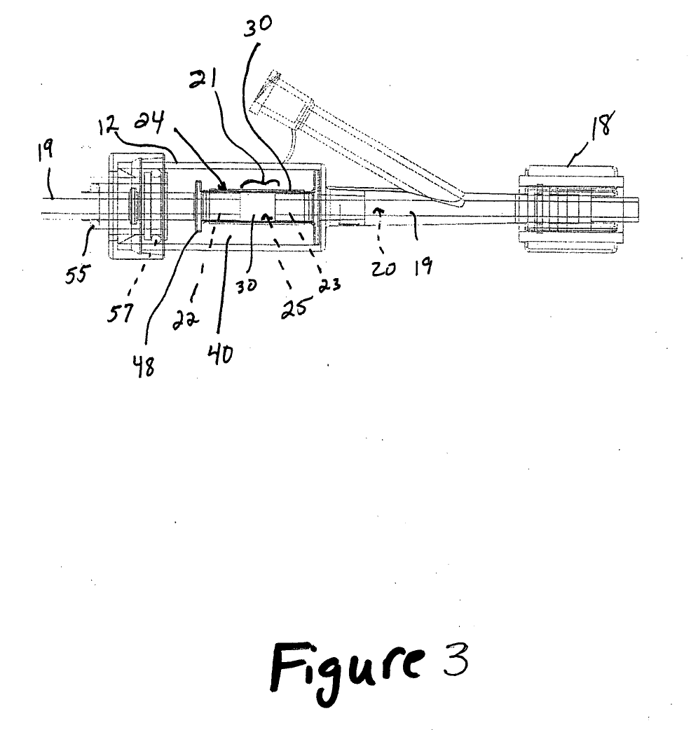

As shown in FIG. 3, the open section 21 has an outer diameter that is substantially the same as the outer diameter of the elongated member 19. Also, the open section 21 has an inner diameter that is greater than the inner diameter of the remaining portions of the through-lumen 20. A collapsible member 24 is secured to the terminal portions 22, 23 and forms a fluid tight relationship with the terminal portions 22, 23 around open section 21, thereby defining chamber 25. The collapsible member 24 also forms a seal around the operating instrument 15 that is positioned within the through-lumen 20 as discussed below.

In a preferred embodiment, the collapsible member 24 includes a collapsible membrane formed of an elastomeric sleeve 30 that is fixedly and sealingly attached to the terminal ends 22, 23. In a preferred embodiment, the sleeve 30 includes a flexible, biocompatible material such as silicone, urethane or latex. However, other materials that are capable of forming a fluid tight seal about the operating instrument 15 can also be used. The sleeve 30 can have an outer diameter of between about 0.125 inch and 0.5 inch, and an inner diameter of between about 0.0625 and 0.4375 inch. In a preferred embodiment, the outer diameter is about 0.1875 inch and the inner diameter is about 0.125 inch. The length of the sleeve 30 (measured between terminal ends 22, 23) is between about 0.25 and 0.50 inch. To facilitate the movement of the operating instrument 15 while maintaining the valve 10 in a closed position, the inner side of sleeve 30 can be coated with a lubricant, such as a hydrogel, to provide a lower friction surface.

The sealing of the sleeve 30 around the inserted surgical instrument 15 can be effected by increasing an existing pressure differential between the chamber 25 and the chamber 40 or by creating a pressure differential between the chamber 25 and the chamber 40. In the embodiments illustrated in FIGS. 1-4, this pressure differential is created by a sealing system 50 that changes the volume within the chamber 40 without permitting fluid to escape from the chamber 40 or the pressure to decrease within the chamber 40 as the sealing system 50 is activated. As a result, the pressure within the chamber 40 increases and the resulting pressure differential between the chamber 40 and the chamber 25 is great enough to create a fluid tight seal around the surgical instrument 15 when the volume in the chamber 40 is reduced by the operation of the sealing system 50.

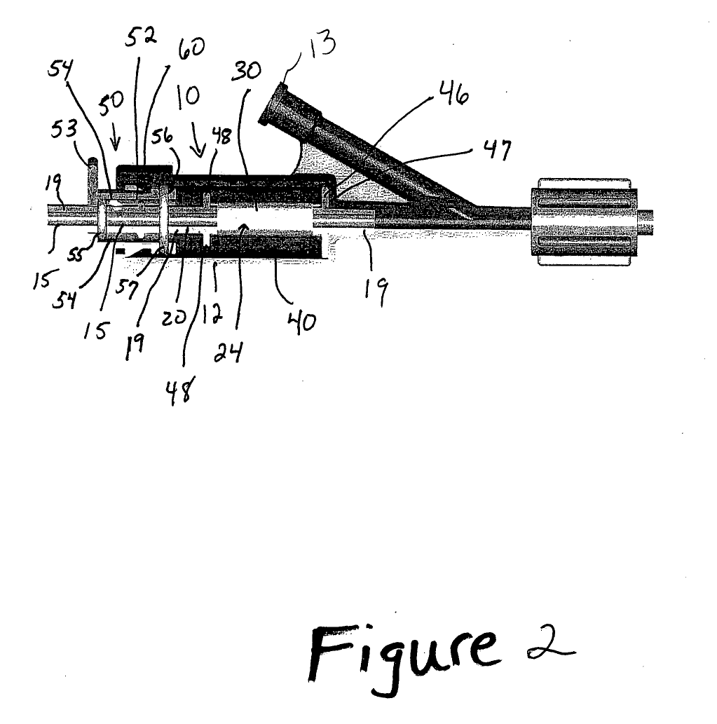

The sealing system 50 includes a moveable plunger (piston) 52 that changes the volume and pressure within the chamber 40 as it moves towards and away from the proximal end 14 of the valve body 12. For example, the pressure within the chamber 40 increases as the plunger 52 moves towards the proximal end 14 (see arrow A in FIG. 1). Similarly, the established pressure within the chamber 40 decreases as the plunger 52 moves away from the open area 21 and toward the distal end 16 (see arrow B in FIG. 1). The plunger 52 includes a disk 53 at a distal end for being pushed or grasped during the operation of the sealing system 50. As shown in FIG. 2, the plunger 52 includes an inner passageway 54 through which the member 19 extends. A sealing member 55 engages with the inner surface of the passageway 54 and an outer surface of the member 19 in order to create a fluid tight seal about the member 19 so that fluids from within the chamber 40 do not leak out or otherwise escape. The outer surface of the plunger includes a groove 56 that carries a sealing member 57 for creating a seal between the outer surface of the plunger 52 and the inner surface of the valve body 12. Like sealing member 55, sealing member 57 prevents fluids from leaking or otherwise escaping from the chamber 40 while the plunger is at rest and as it moves in the direction of the proximal end 14. The sealing members 55, 57 can include rubber O-rings or other conventional sealing rings. Springs can be used to counter the movement of the plunger 52 in the direction of arrow A.

As illustrated in FIGS. 1 and 2, the sealing system 50 also includes a plunger housing 60 positioned on the exterior surface of the valve body. The plunger housing 60 is secured to end of the valve body 12 as shown in FIGS. 1 and 2. Like the plunger 52, the plunger housing 60 also includes a central opening that receives the plunger 52 and the elongated member 19. The plunger housing 60 includes a rear surface 62 for engaging the disk 53 of the plunger 52 to limit the axial movement of the plunger 52 and a front surface 64 that extends away from the valve body 12 and permits the valve 10 to be grasped by a user and operated using only a single hand. As can be understood from the figures, an operator could position two or more of her fingers in front of the front surface 64 and press on the disk 53 of the plunger 52 with her thumb. As a result, one-handed operation of the hemostasis valve according to the present invention is possible.

The elongated member 19 includes a first circumferential stop 46 extending from its outer surface and positioned against an inner end surface 47 of the valve body 12 at the proximal end 14, as shown in FIG. 2, to prevent the elongated member 19 from being unintentionally removed from the interior of the valve body 12. To limit the axial movement of the plunger 52 within the valve body 12, the elongated member 19 also includes a second circumferential stop 48 extending from its outer surface and positioned at a point located between the distal end 16 and the opening 21. As shown, the second circumferential stop 48 is spaced inwardly from the distal end 16. In a preferred embodiment, the distance that the second circumferential stop 48 is spaced from the distal end 16 is the same as the distance from the piston ring 53 to the distal end of the plunger housing 60 when the plunger 52 is at rest.

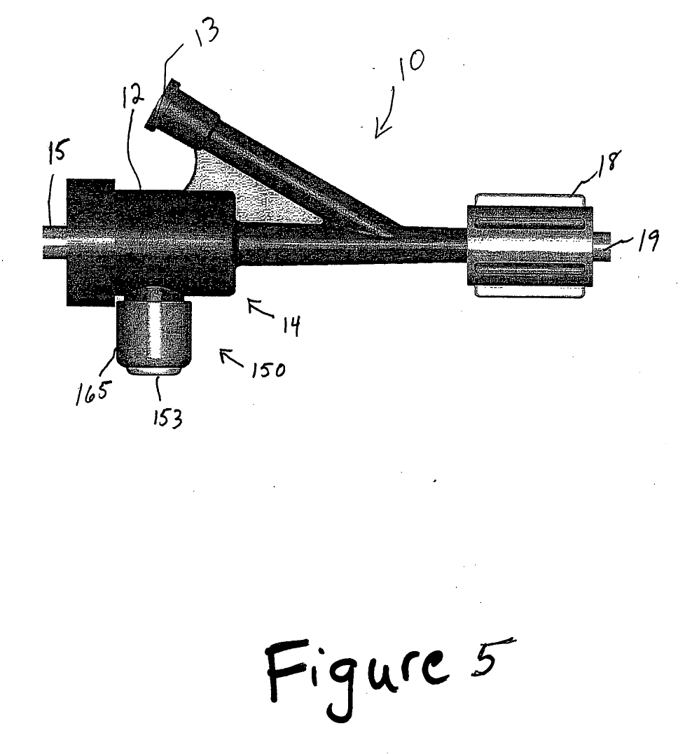

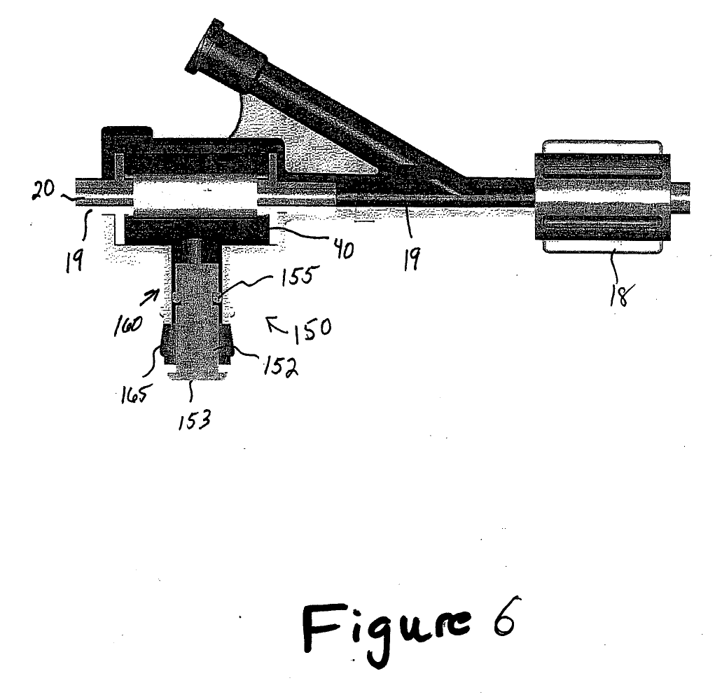

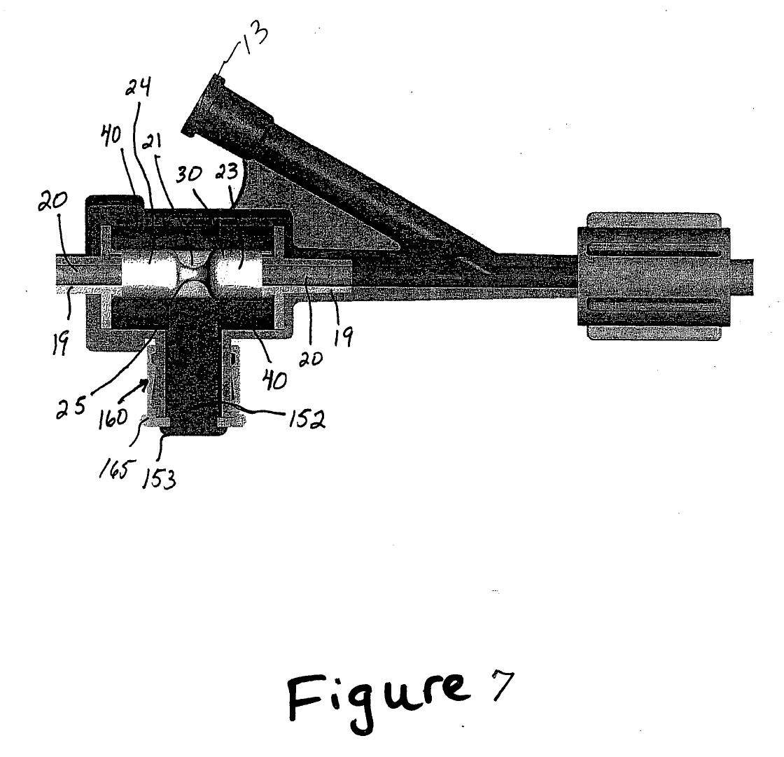

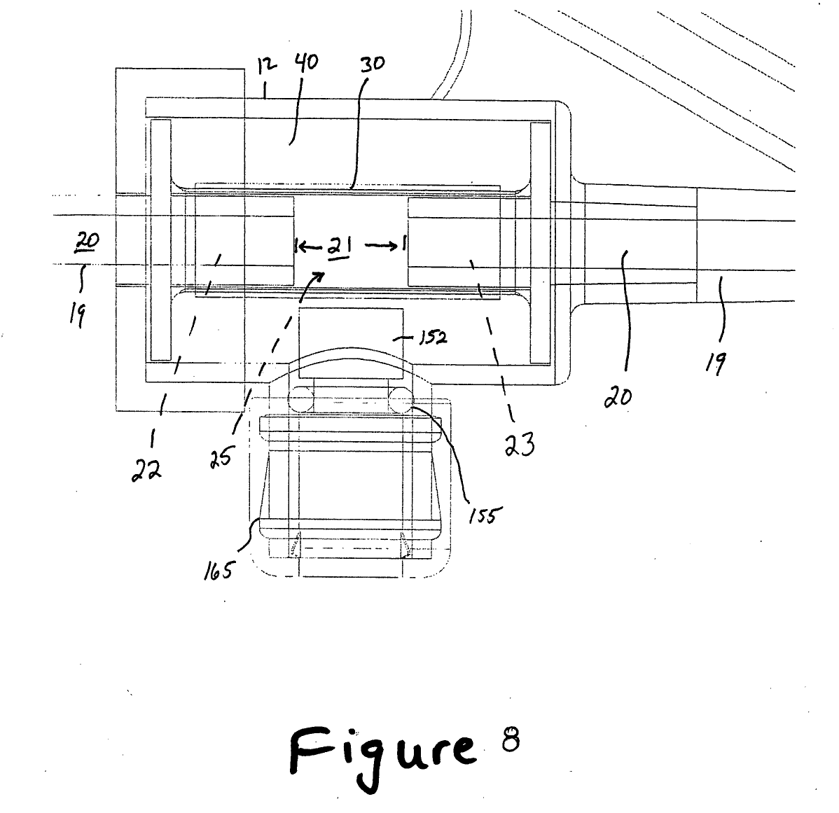

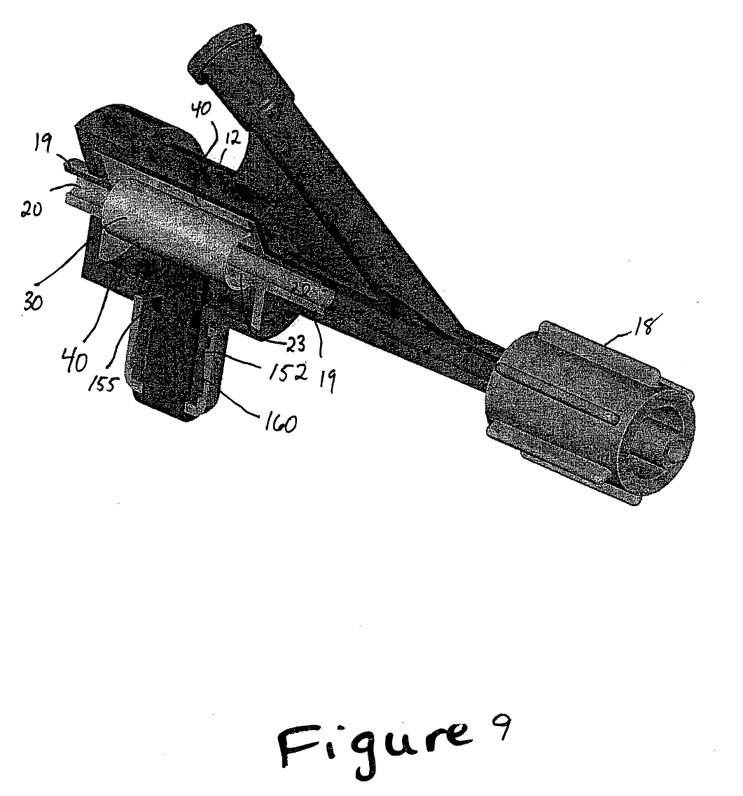

Referring to FIGS. 5-9, an alternative sealing system 150 operates on the same principle as sealing system 50. In sealing system 150, a plunger 152 with a disk 153 is not axially aligned with the elongated member 19 and the instrument 15. Instead, the plunger 152 is positioned in a plunger housing 150 that is transversely aligned with the longitudinal axis of the elongated member 19 as shown in FIGS. 5-7. Additionally, the plunger 152 does not include a central passageway. Instead, in one embodiment, the plunger 152 is solid as shown in FIGS. 6-7. Alternatively, the plunger 152 can have a solid exterior surface that is in communication with the chamber 40 and a hollow, isolated interior. As a result of it solid profile, the plunger 152 only includes one or more sealing members 155 as shown in FIGS. 6-7 for engaging and creating a seal with an inner surface of the port 160. Like sealing members 55, 57, sealing members 155 can include rubber O-rings or similar known circumferential sealing members.

During the operation of each of the above-discussed sealing systems 50, 150, the respective plunger 52, 152 is moved within its housing 60,165 and into the chamber 40 in order to decrease the volume of the chamber 40 and increase the pressure within the chamber 40. As discussed above, no pressure or fluid is released from the chamber 40 during the movement of a respective plunger 52, 152. The pressure increase within the chamber 40 causes the elastomeric sleeve 30 to collapse around the medical instrument 15 and create a seal.

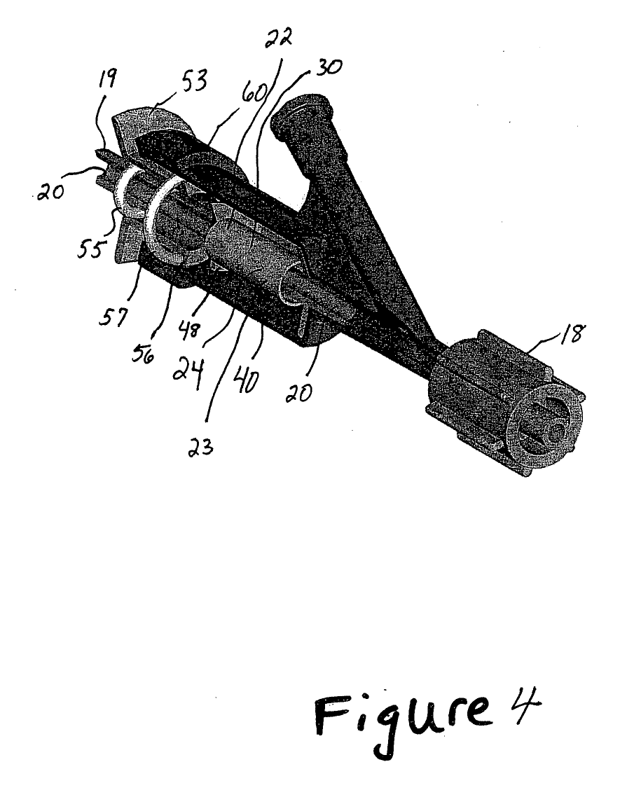

During the operation, the plunger 52, 152 moves along a path of motion from its rest position, as shown in FIGS. 1 and 6, respectively, to its final pressure increasing position, as shown in FIGS. 4 and 7, respectively. The plungers 52, 152 move from their rest position toward the final pressure position when pushed. As a result, the plunger 52, 152 can stop at an infinite number of locations along its path of motion. Therefore, the pressure within the chamber 40 can experience an infinite number of increases. The stops, such as circumferential stop 48, limit the movement of the plungers 52, 152 when they reach the end of their paths of travel. As shown in the figures, the volume of the chamber 40 is smaller when the plungers 52, 152 are at the end of their travel paths then when they assume their rest positions.

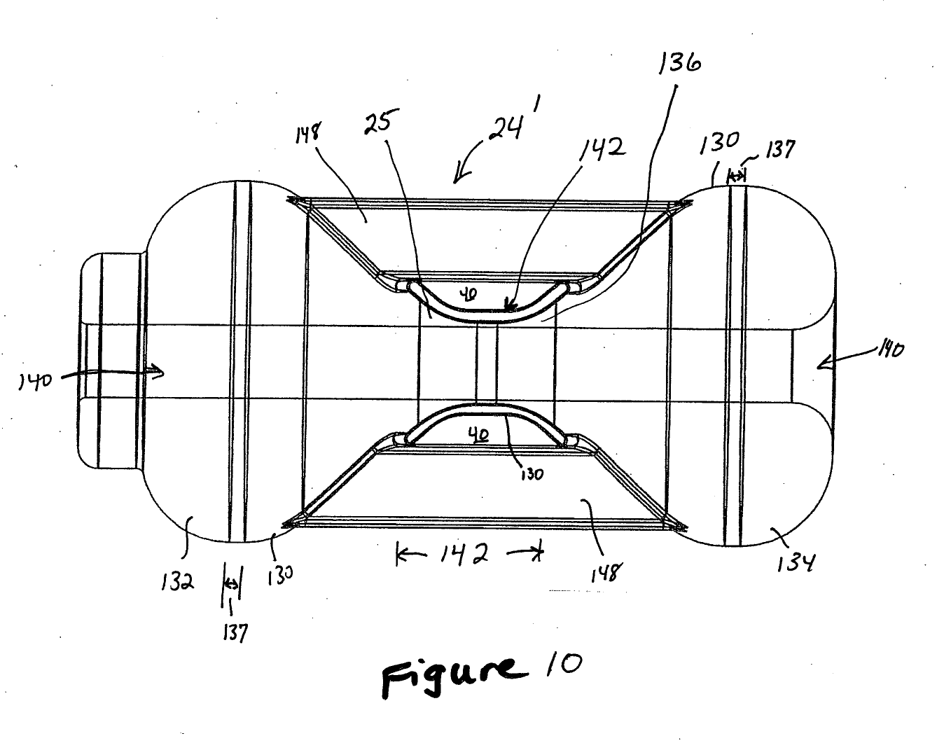

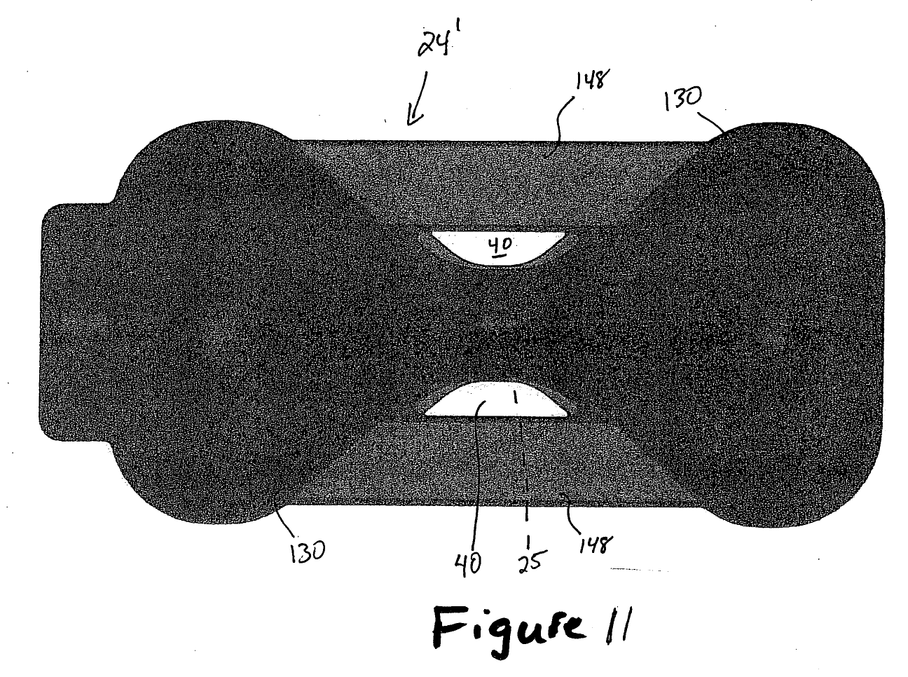

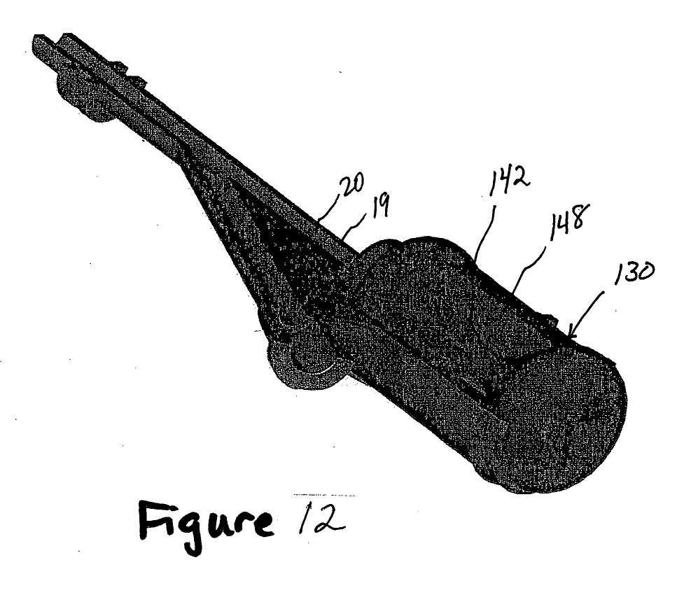

In an additional embodiment illustrated in FIGS. 10-12, the collapsible member 24′ within the valve body 12 includes a flexible member 130 that operates substantially the same as flexible member 30 and can be used with any of the above-discussed embodiments. For example, the flexible member 130 is positioned within the outer chamber 40 and defines the inner chamber 25. A plunger such as plunger 52 can be introduced from the left side of FIG. 10 as discussed above with respect to the embodiment illustrated in FIG. 1. Additionally, the flexible member 130 collapses in response to increased pressure within the outer chamber 40 as does flexible member 30. As shown in FIG. 10, the flexible member 130 has an inner passageway 140 that seals with the member 19 and has a section 142 that seals around the inserted medical instrument 15. Additionally, the flexible member 130 includes two spaced support members 148 that box the flexible member 130 and prevent it from loosing its external shape in response to a pressure increase within chamber 40. These boxing support members 148 also allow the collapsible section 142 to form a seal with the inserted medical instrument while preventing the flexible member 130 from collapsing.

In addition to forming a seal about an inserted medical instrument 15 in response to an increase in pressure within the chamber 40, the flexible member 130 also forms a seal with the inner surface of the valve body 12 housing. The flexible member 130 includes a first bulbous section 132, a second bulbous section 134 and a central connecting section 136 that extends between the bulbous sections 132, 134. Each bulbous section 132, 134 has a region 137 that contacts the inner surface of the valve body 12 and forms a fluid tight seal within the valve body 12. As a result, sealing members are not needed to maintain the pressure within the outer chamber 40 when the plunger 52 moves from the rest position toward its final sealing position.

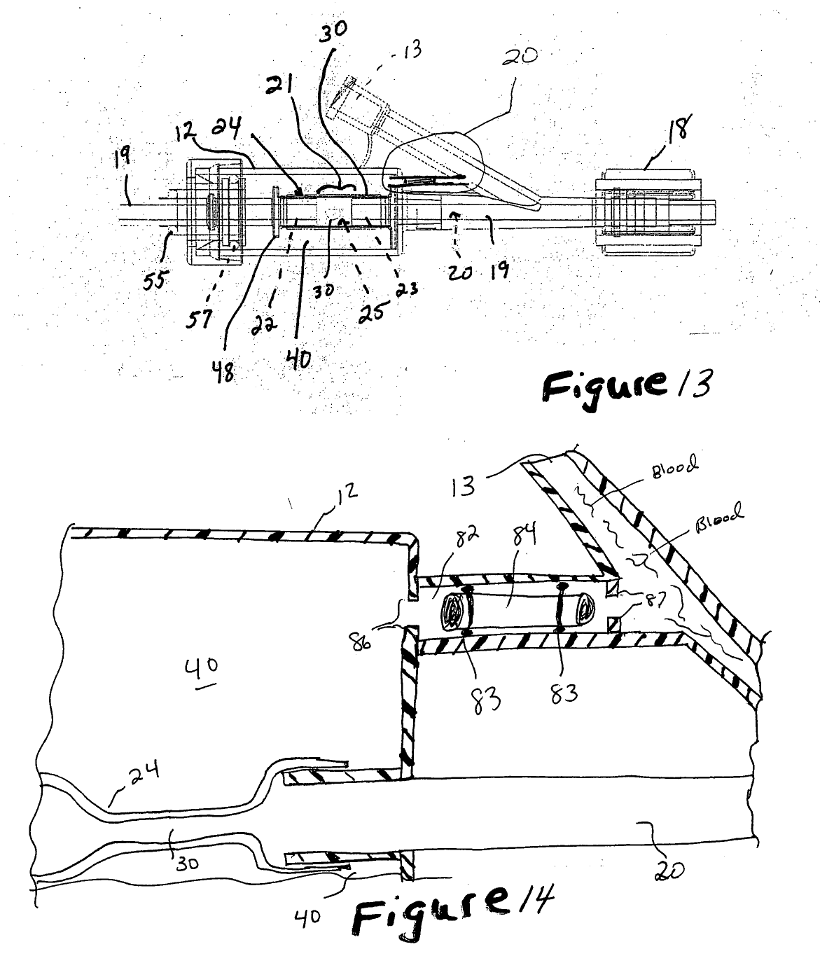

As illustrated in FIGS. 13 and 14, the above-discussed embodiments of the hemostasis valve 10 according to the present invention can also include a system for increasing or decreasing the pressure within the outer chamber 40 in response to a pressure increase (blood pressure) within the catheter system that occurs during injections of contrast or saline. The valve 10 can include a hollow lumen 82 extending between the injection port 13 and the chamber 40. A solid sliding member 84 carrying two sealing members 83, for example O-rings, is free to move toward or away from the injection port 13 in response to the blood pressure within the injection port 13. Stops 86, 87 are provided for limiting the travel of the sliding member 84. A precharge in chamber 40 or bellows may be used to assist pressure increases within chamber 40 in response to blood pressure increases.

Additionally, the above-discussed hemostasis valves can also include a system for continuous flushing the attached guide catheter and bellows that act as an expandable fluid reservoir as disclosed in U.S. Pat. No. 5,895,376 to Schwartz et al., which is hereby fully incorporated herein by reference.

It should be understood that the present invention is not limited to the preferred embodiments discussed above which are illustrative only. Changes may be made in detail, especially in matters of shape, size, arrangement of parts, or material of components within the principles of the invention to the full extent indicated by the broad general meanings of the terms in which the appended additional features are expressed.

1. A hemostasis valve comprising:
a valve body having a proximal end for connecting to a first medical device and a distal end for connecting to a second medical device;
a first elongated chamber within said valve body, said first chamber having a first internal volume and being capable of receiving a medical instrument therein;
a collapsible member positioned within said valve body and defining said first elongated chamber;
a second elongated chamber extending about said first elongated chamber within said valve body, said second elongated chamber having an internal volume that is greater than said first internal volume; and
a pressure application system comprising a member moveable within said second elongate chamber for increasing the pressure within said second elongate chamber and sealing said collapsible member about a received medical instrument.

2. The hemostasis valve of claim 1 wherein said moveable member includes a plunger that is moveable relative to the valve body for reducing the interior volume within said second elongated chamber.

3. The hemostasis valve of claim 2 wherein a longitudinal axis of said plunger extends substantially parallel to a longitudinal axis of said valve body.

4. The hemostasis valve of claim 2 wherein a longitudinal axis of said plunger extends substantially perpendicular to a longitudinal axis of said valve body.

5. The hemostasis valve of claim 1 wherein said pressure application system further comprises a housing for said moveable member, said moveable member including a sealing member for cooperating with an inner surface of said housing to create a fluid tight seal between said housing and said moveable member.

6. The hemostasis valve of claim 5 wherein said moveable member includes an internal lumen for receiving an elongated member capable of carrying a medical instrument, said internal lumen includes a seal for creating a fluid tight seal with said elongated member.

7. The hemostasis valve of claim 6 wherein said elongated member includes an interior lumen aligned with the first chamber for receiving the medical instrument.

8. The hemostasis valve of claim 1 wherein moveable member can provide an infinite amount of pressure adjustments within said second elongated chamber.

9. The hemostasis valve of claim 1 wherein said second elongated chamber has a substantially hour glass shape.

10. The hemostasis valve of claim 9 wherein said valve body includes a housing, and wherein said substantially hour glass shaped member includes first and second bulbous sections that form a seal with an inner surface of the valve body housing.

11. A method of sealing a hemostasis valve about a medical instrument, said method comprising the steps of:
positioning a medical instrument within a first chamber in a valve body of the hemostasis valve;
advancing a pressure increasing element within a second chamber of said valve body, said second chamber surrounding at least a portion of said first chamber.
